(19) United States
(12) Patent Application Publication
(10) Pub. No.: US 2011/0038938 A1
(43) Pub. Date: Feb. 17, 2011

(54) Compositions for tissue augmentation

(76) Inventors: Ira C. Ison, San Jose, CA (US); Brooke Campbell, Austin, TX (US)

Correspondence Address: PETERS VERNY , L.L.P., 425 SHERMAN AVENUE, SUITE 230, PALO ALTO, CA, 94306, US

(21) Appl. No.: 12/859,004

(22) Filed: Aug. 18, 2010

Related U.S. Application Data
(63) Continuation of application No. PCT/US2009/034640, filed on Feb. 20, 2009.
(60) Provisional application No. 61/030,812, filed on Feb. 22, 2008.

(51) Int. Cl. A61K 9/14; A61K 47/02; A61K 47/34; A61K 47/36; A61K 47/38; A61K 47/42; A61K 31/192; A61K 31/167; A61K 31/56; A61K 38/18; A61K 31/616; A61K 31/573; A61P 29/00; A61P 23/00; A61P 31/00

(52) U.S. Cl. 424/489; 514/770; 514/772.3; 514/777; 514/781; 514/778; 514/772; 514/773; 514/774; 514/570; 514/626; 514/169; 514/8.8; 514/7.6; 514/165; 514/180

(57) ABSTRACT

The embodiments set forth herein provide biocompatible self-setting compositions suitable for use in tissue augmentation applications. The biocompatible self-setting compositions described herein exhibit advantageous theological properties and may be applied to a site in the body of a patient by injecting the composition through a 20-30 gauge needle. Once applied to a site in the body, the composition sets to a slow resorbing or substantially non-resorbing matrix. Advantageously, exposure of the composition material to body heat at its site of use enhances setting of the composition. Composition materials prepared in accordance with the present disclosure may find use in applications involving, for example, soft tissue augmentation such as for dermal fold augmentation, prevention of adhesions, soft tissue void filling, soft tissue bleb creation, urethral sphincter augmentation for treatment of urinary incontinence, treatment of unilateral vocal fold paralysis, and lower esophageal sphincter augmentation for treatment of gastroesophageal reflux disease. In some embodiments, the presently described biocompatible compositions may serve as bone void fillers.

### BACKGROUND OF THE INVENTION

1. Field of the Invention

The invention relates to biocompatible self-hardening compositions suitable for use as dermal fillers. More specifically, the invention relates to composition that can be used for soft tissue augmentation such as for dermal fold augmentation, prevention of adhesions, soft tissue void filling, soft tissue bleb creation, urethral sphincter augmentation for treatment of urinary incontinence, treatment of unilateral vocal fold paralysis, and lower esophageal sphincter augmentation for treatment of gastroesophageal reflux disease. Secondarily, these biocompatible compositions may serve as bone void fillers.

2. Description of the Relevant Art

Examples of materials used in previous inventions for the same purposes and utilities of this invention include, for example, collagen, hyaluronic acid, hydroxyapatite, dextran, poly-L-lactic acid, polyvinyl alcohol, chitosan, hydroethylmetacrylate-ethylmetacrylate copolymer, polymethylmethacrylate, polyacrylamide, polyacrylonitrile, polyalkylimide, polytetrafluoroethylene, polydimethylsiloxane, sodium carboxymethylcellulose, hydroxypropylmethylcellulose, etc. These chemicals are manufactured in the form injectable biomaterials which sometimes utilize an additional lubricant, such as water, glycerol, oils, polysaccharides, starch, etc. to allow for the degree of injectability necessary for the augmentation procedures.
U.S. Pat. Nos. 7,060,287, 6,558,612, 6,537,574, 6,432,437, and 5,922,025 by Hubbard et al claim low solubility ceramic spheres (preferably hydroxyapatite) greater than 20 microns suspended in a polysaccharide gel (preferably sodium carboxymethylcellulose) for an injectable biomaterial in tissue augmentation.
U.S. patent applications 20040185021 and 20020151466 by Hubbard claim an injectable tissue augmentation biomaterial comprised of polysaccharide gel selected from the group consisting of a cellulose polysaccharide, starch, chitin, chitosan, hyaluronic acid, hydrophobe modified polysaccharide, an alginate, a carrageenan, agar, agarose, an intramolecular complex of a polysaccharide, an oligosaccharide and a macrocylic polysaccharide. Glycerin may also be included.
U.S. Pat. No. 7,192,984 by Berg et al claims carboxymethylcellulose with polyethylene oxide and calcium ions for dermal filler.
U.K. patent 2227176 by Ersek et al, claims microparticles of polytetrafluoroethylene, polydimethylsiloxane, and inert ceramics with fluids.
U.S. Pat. No. 4,803,075 by Wallace et al claims polymer particulates in water.
U.S. Pat. No. 4,837,285 by Berg et al claims porous crosslinked collagen heads.
U.S. Pat. No. 4,280,954 by Yannas claims crosslinked collagen and mucopolysaccharide
U.S. Pat. No. 4,352,883 by Lim claims a method to encapsulate living tissue with polysaccharide gels crosslinked with pH change or multivalent cations such as calcium.
U.S. Pat. No. 7,265,098 and 6,566,345 by Miller et al claim polysaccharides and polyethers in foams to prevent adhesions.
U.S. Pat. No. 6,923,961 by Liu et al claims claims carboxymethylcellulose in solids and gels for antiadhesion, antithrombogenic, drug delivery, and hemostasis.
U.S. Pat. No. 6,869,938, 6,133,325, 6,034,140, and 6,017,301 by Schwartz et al claim carboxyl-containing polysaccharides, polyethers, polyacids, polyalkylene oxides, multivalent cations and polycations in solids and gels for antiadhesion
U.S. Pat. No. 6,743,775 by Santar et al claims slow release formulations using anionic polysaccharides.
U.S. Pat. No. 6,432,449 by Goldenberg et al claims sustained release formulations using alginate gel.
U.S. Pat. No. 6,409,881 by Jaschinski claims cellulosic compounds ionically crosslinked with transition metals.

The aforementioned prior art references are incorporated by reference as though fully set forth herein.

### SUMMARY OF THE INVENTION

The embodiments set forth herein provide biocompatible self-setting compositions suitable for use in tissue augmentation applications. The biocompatible self-setting compositions described herein exhibit advantageous rheological properties and may be applied to a site in the body of a patient by injecting the composition through a 20-30 gauge needle. Once applied to a site in the body, the composition sets to a substantially non-resorbing matrix. Advantageously, exposure of the composition material to body heat at its site of use may enhance setting of the composition. Composition materials prepared in accordance with the present disclosure may find use in applications involving, for example, soft tissue augmentation such as for dermal fold augmentation, prevention of adhesions, soft tissue void filling, soft tissue bleb creation, urethral sphincter augmentation for treatment of urinary incontinence, treatment of unilateral vocal fold paralysis, and lower esophageal sphincter augmentation for treatment of gastroesophageal reflux disease. In some embodiments, the presently described biocompatible compositions may serve as bone void fillers.

In an embodiment, a biocompatible self-setting composition may include cement particles capable of undergoing a cementing reaction when contacted with a suitable setting liquid. The cement particles may be dispersed throughout a crosslinkable polymer gel. Individual molecules within the polymer gel may be crosslinked by exposing the polymer gel to multivalent cations. The self-setting composition may exhibit improved setting characteristics at body temperature.

In an alternate embodiment, a biocompatible self-setting injectable composition suitable for use as a dermal filler may include a poorly soluble source of inorganic ions in combination with at least one crosslinkable polymer gel. The polymer gel is capable of undergoing ionic crosslinking in the presence of multivalent ions. The self-setting composition may exhibit improved setting characteristics at body temperature.

In a further alternate embodiment, a self-setting injectable polymeric composition may include at least one crosslinkable polymer gel in combination with at least one covalent or ionic crosslinking agent. The covalent or ionic crosslinking agent is mixed with the crosslinkable polymer gel substantially immediately prior to injection of the polymeric composition, thereby allowing crosslinking of the polymer gel in situ.

In yet a further alternate embodiment, a self-setting injectable polymeric composition may include at least one crosslinkable polymer gel and at least one covalent or ionic crosslinking agent. The covalent or ionic crosslinking agent may be mixed with a biocompatible time delay release agent prior to addition thereof to the gel polymer. Delayed release of the covalent or ionic crosslinking agent from the biocompatible time delay release agent may allow for crosslinking of the polymer gel composition in situ.

### DETAILED DESCRIPTION OF THE PREFERRED EMBODIMENTS

### Definitions

In order to facilitate understanding of the invention, a number of terms are defined below. It will further be understood that, unless otherwise defined, all technical and scientific terminology used herein has the same meaning as commonly understood by practitioners of ordinary skill in the art to which this invention pertains.

As used herein, the term “therapeutic agent” generally refers to a composition that is capable of inducing or affecting an action in a biological system, e.g. by inducing or affecting a therapeutic or prophylactic effect, an immune response, tissue growth, cell growth, cell differentiation or cell proliferation. A therapeutic agent may include a pharmaceutical delivery vehicle. The delivery vehicle would typically be optimized to stably accommodate an effective dosage of one or more compounds having biological activity. The determination of the effective dose of a therapeutic agent that should be included in a bioactive composition to achieve a desired biological response is dependent on the particular compound, the magnitude of the desired response, and the physiological context of the composition. Such determinations may be readily made by an ordinary practitioner of the pharmaceutical arts, Components of therapeutic agent may include growth factors, analgesics, antibiotics, or other pharmacologically active compounds.

As used herein, the term “antibiotic” generally refers to a naturally occurring, synthetic or semi-synthetic chemical substance that is derivable from a mold or bacterium that, when diluted in an aqueous medium, kills or inhibits the growth of microorganisms and can cure or treat infection.

As used herein, the term “analgesic” is used in reference to a pharmacologically active agent or composition that alleviates pain without causing loss of consciousness.

The term “ionic crosslinking” as used throughout the disclosure refers to process whereby functional groups present on individual gel polymer molecules form ionic interactions with multivalent cations present in the surrounding medium thereby creating a substantially continuous gel polymer matrix. The term “ionic crosslinking” specifically excludes covalent crosslinking of gel polymer molecules, as described below in an alternate embodiment.

The present disclosure describes preparation and use of permanent and semi-permanent biocompatible compositions for soft and hard tissue augmentation. The compositions include self setting biocompatible cements or salts in a composition together with biocompatible polymer gels. The composition combinations set in vivo to form biocompatible composition materials capable of providing a scaffold supporting local autogenous, non-scar soft or hard tissue growth.

The cement portion of the composition serves as a slow (or non-) resorbing mat ix aiding in the longevity of the duration of augmentation. Suitable cements may include various ionic compounds containing cations such as calcium, magnesium, strontium, sodium, potassium, barium, lithium, aluminum, iron, copper, manganese, chromium, zinc, etc, combined with anion groups (fully or partially neutralized) such as phosphate (and acid phosphates), sulfates, oxide, carbonate (and bicarbonate), chlorides, borates, etc. Preferred cement compounds include any combinations of calcium phosphate cements, magnesium phosphate cements, strontium phosphate cements, calcium aluminate cements, calcium sulfate cements, and calcium silicate aluminate cements (such as for use in ionomer-type cements). The following calcium phosphate cements, as well as methods of making and using same, are provided by way of non-limiting example only. It will be readily appreciated by the skilled artisan that any art-recognized cement may be used, Calcium phosphate cements suitable for use with the presently described embodiments may include, without limitation, those calcium phosphate cements, and methods of making same, disclosed in U.S. Pat. Nos. 6,379,453 and 6,840,995 to Lin et al., entitled “PROCESS FOR PRODUCING FAST SETTING, BIORESORBABLE CALCIUM PHOSPHATE CEMENT”, U.S. Pat. No. 6,616,742 to Lin et al. entitled “PROCESS FOR PREPARING A PASTE FROM CALCIUM PHOSPHATE CEMENT”, U.S. Pat. No. 6,648,960 to Lin et al entitled “METHOD OF SHORTENING A WORKING AND SETTING TIME OF A CALCIUM PHOSPHATE CEMENT (CPC) PASTE”, U.S. Patent Application Publication Nos. 2004/0031420 to Lin et al. entitled “CALCIUM PHOSPHATE CEMENT, USE AND PREPARATION THEREOF,” 2004/0003757 to Lin et al. entitled “TETRACALCIUM PHOSPHATE (TTCP) HAVING CALCIUM PHOSPHATE WHISKER ON SURFACE,” 2005/0076813 to Lin et al. entitled “PROCESS FOR PRODUCING FAST-SETTING BIORESORBABLE CALCIUM PHOSPHATE CEMENT,” 2005/0069479 to Lin et al. entitled “METHOD OF INCREASING WORKING TIME OF TETRACALCIUM PHOSPHATE CEMENT PASTE,” 2005/0184417 to Lin et al. entitled “METHOD FOR MAKING A POROUS CALCIUM PHOSPHATE ARTICLE,” and in U.S. patent application Ser. No. 10/773,701 to Lin et al. entitled “TETRACALCIUM PHOSPHATE (TTCP) HAVING CALCIUM PHOSPHATE WHISKER ON SURFACE AND PROCESS FOR PREPARING THE SAME,” U.S. patent application Ser. No. 10/633,511 to Lin et al. entitled “METHOD OF MAKING A MOLDED CALCIUM PHOSPHATE ARTICLE,” and Int'l Patent Appl. Publ. No. WO 2005/016616 to Lin et al. entitled “METHODS FOR PREPARING MEDICAL IMPLANTS FROM CALCIUM PHOSPHATE CEMENT AND MEDICAL IMPLANTS.” The above-cited patent references are hereby incorporated by reference in their entirety as though fully sot forth herein. In an embodiment, the average diameter of cement particles may be less than 150 μm, less than 100 m, or less 90 μm.

Suitable polymer components may include any combination of biocompatible polymers which aid in any of the following: (1) serve as lubricious carriers and dispersants to improve the ease of mixing and delivery of the augmentation material prior to set; (2) help to prevent dispersion of cement particulate; (3) aid in creating porosity for tissue ingrowth; (4) form an in situ setting composition with the cement component; and (5) adjust the final physical properties of the set composition where the final material would have greater viscosity or elasticity than the original material prior to injection or implantation. Also, some of the polymeric components preferably are hydrogels, or miscible in water, or may be emulsified in water such that the cement may set in an aqueous environment. Also, some of the polymeric components preferably are chosen such that, in theory, they can interact with divalent and trivalent cations and can be ionically crosslinked such that in situ crosslinking may occur serving to increase the duration of augmentation. Also, preferred polymers include those that may be crosslinked in situ by change or adjustment of some combination of ionic crosslinking, pH, and temperature. The theory of ionic crosslinking as well as other methods of crosslinking only serve to account for observations and aid in selection of the polymers, and the invention is not intended to be limited to any particular theory.

Exemplary though non-limiting gelling polymers and other time release compounds suitable for use in accordance with the present embodiments include polysaccharide gels such as gels made with water and any combination of the following: cellulose, agarose, agar, agar methylcellulose, hydroxypropyl cellulose, hydroxypropyl methylcellulose, ethyl cellulose, carboxyethyl cellulose, microcrystalline cellulose, oxidized cellulose, sodium carboxymethylcellulose, dextran, carboxymethyl dextran, chitosan, chitin, carboxymethyl chitin, hyaluronic acid, sodium hyaluronate, pectin, alginate, carrageenan, and starch. Other anionic polysaccharides include polyuronic acids and their biocompatible salts and copolymers such as polymannuronic acid, polyglucuronic acid, polyanhydroglucuronic acid, and polyguluronic acid. Glycosaminoglycans such as heparin, heparin sulfate, and chondroiton sulfate may be used. Additional polymers which may be incorporated in this invention are polyalkalene oxides and poly acids such as polyethylene glycol, polyethylene oxide, polypropylene glycol, polypropylene oxide, propylene glycol alginate, polyacrylates and their acids, polylactates and their acids, polyglycolates and their acids, polymethacrylates and their acids, polymethylmethacrylates and their acids, polyterephthalic acid, polyhydroxybutyric acid, polyphosphoric acid, polystyrenesulfonic acid, polyamino acids, and various nonionic block copolymers such as poloxamers. Pluronics (a.k.a. poloxamers) are of interest due to their thermoreversible nature which demonstrate phase transition between ambient room temperature and physiological temperature in vivo. Other polymers which exhibit thermoreversible characteristics and are included in this invention are naturally occurring polymers such as gelatin (a protein prepared from partial hydrolysis of collagen), polysaccharides such as agarose extracted from red seaweed, alternating copolymer of 1,4-linked 3,6-anhydro-a-L-galactose and 1,3-linked b-D-galactose), amylose (1,4-linked a-D-glucan linear polymer), amylopectin (a-1,6 glucan with a large number of a-1,4-glucan branches), carrageenans (extracted from red seaweed, alternating copolymer of 1,4-linked a-D-galactose and 1,3-linked b-galactose, containing ester sulfate), and Gellant (Kelco Division of Merck&Co., CA) (polysaccharide originated from bacteria, consisting of b-glucose-b-D-glucuronic acid-b-glucose-a-Lrhamnose). Other cationic components of interest include for example derivatives and copolymers of acrylamide, methacrylamide, butylacrylate, maleinanhydride, and methylmethacrylate such as polyacrylamide, hydroxyethylmethacrylate, hydroxypropylmethacrylamide. Peptides such as gelatine, protamine, and fibrinopeptide also may be utilized as cations. For the purpose of increasing implant longevity, this invention also may include non-resorbable biocompatible polymers such as polyethylene, polypropylene, fluoropolymers (e.g. polytetrafluoroenthylene, perfluoroalkoxy, fluorinated ethylene propylene), polymerized methyl-, ethyl-, and phenol-siloxanes, polyetheretherketone, and any biocompatible polymers known in the art which are suitable for making gels or solid microspheres.

An alternate embodiment of the present invention includes a self setting biocompatible composition of inorganic ions which, when combined with polymer gels, the composition combinations set in vivo to form biocompatible composition materials of inorganic salts with low solubility in combination with gels capable of providing a scaffold supporting local autogenous, non-scar soft or hard tissue growth. This material would have greater viscosity or elasticity than the original material prior to injection or implantation.

The inorganic ions of the composition serve as a slow (or non-) resorbing matrix aiding in the longevity of the duration of augmentation. Examples of suitable ions may include cations such as calcium, magnesium, strontium, sodium, potassium, barium, lithium, aluminum, iron, copper, manganese, chromium, zinc, etc. combined with anion groups (fully or partially neutralized) such as phosphate (and acid phosphates), sulfates, oxide, carbonate (and bicarbonate), chlorides, borates, etc. Examples of suitable polymer gels include for example the same as listed above for the compositions containing cements and polymers.

In yet a further alternate embodiment of the present invention, a self setting (or gelating) biocompatible polymeric cement-like compound involving biocompatible polymer gels which may be crosslinked in situ and made to set in vivo to form a biocompatible material. Such material may have greater viscosity or elasticity than the original material prior to injection or implantation. This material may be capable of providing a scaffold supporting local autogenous, non-scar soft or hard tissue growth. The in situ crosslinking reaction may be accomplished by methods similar to those used in the art of polymeric crosslinking reactions. For example, covalent or ionic crosslinking post-implantation can be accomplished by mixing multiple chemical components just prior to or during injection or implantation, which result in delayed crosslinking with sufficient working time for the operative procedure. This delay is achieved by adjusting the rate of the crosslinking reaction. Another approach is the use of a biocompatible time delay release agent, such as an excipient or porous material or other material capable of releasing reactants. The reactants involved in crosslinking are contained within the time delay release agent, and hence delays the contact between one or more reactants until after injection or implantation. Such a time-release compound can be selected from those used in the art. Examples of rapidly soluble excipients include some polyols (e.g. xylitol, mannitol, sorbitol, isomalt, maltitol, lactitol, etc.), sugar saccharides (e.g. lactose, maltose, trehalose, sucrose, dextran, etc.), and any biocompatible excipients which are not prone to excessive ionic crosslinking. Examples of other biocompatible time release agents include slowly soluble or non-resorbing insoluble porous compounds including for example biodegradable polymers (e.g. polylactide, polyglycolide, PLGA, polycaprolactone, polydiaxanone, polytrimethylene carbonate, etc.). Examples of possible reactants are compounds listed below for achieving a crosslinked polymer.

Preferred oligomers and polymers for in situ setting compounds include polyols for dispersants, thickeners, and time release agents such as glycerol, glycol, erythritol, arabitol, xylitol, mannitol, sorbitol, isomalt, maltitol, lactitol, and polyvinyl alcohol. Mono and disaccharides also may be used in this invention for the same purposes.

In an embodiment, a method to control and adjust pH, ion release rate, common ions, and net ionic charge in this invention for the purposes of adjusting rheology, setting time, and crosslinking may include the use of soluble or partially soluble acids, bases, and salts. Preferred salts, acids, and bases (to include partially neutralized acid salts and hydrated salts) include biocompatible inorganic compounds of anions (e.g. phosphates, chlorides, sulfates, carbonates, ammoniums, oxides, and hydroxides) neutralized with metal cations (e.g. sodium, potassium, calcium, magnesium, strontium, barium, lithium, beryllium, aluminum, iron, hydrogen). Also included in this invention are polycations and polyanions (which may be used, for example, to aid with crosslinking, pH adjustment, rheology, and setting time). Such compounds include, for example, mono-, di-, and tricarboxylic acids and their salts (e.g. citric, acetic, acrylic, malonic, fumaric, malic, maleic, formic, propionic, butyric, valeric, caproic, enanthic, caprylic, peargonic, capric, lauric, stearic, lactic, glycolic, tartaric, gluconic, glucuronic, etc.) which may be neutralized with metals (e.g. sodium, potassium, calcium, magnesium, strontium, barium, lithium, beryllium, aluminum, iron, etc.). Other polycations and polyanions (which may be used, for example, to aid with adjustment of crosslinking, rheology, and setting time) include polylysine, polyarginine, chitosan, and any other biocompatible monomer, dimer, and polymer compounds containing net positive or negative charges under aqueous conditions.

The compositions claimed may incorporate drugs, adjuvants, and other medicaments to be delivered to the injection or implantation site. The incorporation of such drugs, etc. may be incorporated into one of the components of the invention during manufacturing or may be mixed in at the time of implantation. Such drugs may include for purpose of example analgesics and anesthetics (such as lidocaine, etc.), anti-inflammatories (such as ibuprofen, ketoprofen, aspirin, etc.), steroids (such triamcinolone, etc.), antibiotics (such as tetracycline, etc.), antihistamines, and synthetic and autologous soft and hard tissue inductive growth factors (such as fibroblast growth factor (FGE), epidermal growth factor (EGF), platelet derived growth factor (PDGF), bone morphogenic proteins (BMP), etc.). Also, chemotherapy agents (such as alkylating agents including cisplatin, etc.) may be incorporated into this invention. Further, neurotoxic analgesics (such botulinum protein, etc.) also may be incorporated into the compositions of this invention.

The compositions of this invention can be injected intradermally or subcutaneously or can be surgically implanted. The compositions of this invention can be separate components manufactured separately and mixed just prior to injection or implantation or during injection or implantation. Additionally any components may be mixed sequentially in a sequence which allows the proper function of the final material. This invention includes the separate components to be mixed together in any appropriate order to create the final product. This invention also includes the mixture of the components as well as the resultant materials in vivo just prior to, during, and after curing to its final composition.

Calcium phosphate cements suitable for use with the presently described embodiments may include, without limitation, those calcium phosphate cements, and methods of making same, disclosed in U.S. Pat. Nos. 6,379,453 and 6,840,995 to Lin et al., entitled “PROCESS FOR PRODUCING FAST SETTING, BIORESORBABLE CALCIUM PHOSPHATE CEMENT”, U.S. Pat. No. 6,616,742 to Lin et al. entitled “PROCESS FOR PREPARING A PASTE FROM CALCIUM PHOSPHATE CEMENT”, U.S. Pat. No. 6,648,960 to Lin et al entitled “METHOD OF SHORTENING A WORKING AND SETTING TIME OF A CALCIUM PHOSPHATE CEMENT (CPC) PASTE”, U.S. Patent Application Publication Nos. 2004/0031420 to Lin et al. entitled “CALCIUM PHOSPHATE CEMENT, USE AND PREPARATION THEREOF,” 2004/0003757 to Lin et al, entitled “TETRACALCIUM PHOSPHATE (TTCP) HAVING CALCIUM PHOSPHATE WHISKER ON SURFACE,” 2005/0076813 to Lin et al. entitled “PROCESS FOR PRODUCING FAST-SETTING BIORESORBABLE CALCIUM PHOSPHATE CEMENT,” 2005/0069479 to Lin et al. entitled “METHOD OF INCREASING WORKING TIME OF TETRACALCIUM PHOSPHATE CEMENT PASTE,” 2005/0184417 to Lin et al. entitled “METHOD FOR MAKING A POROUS CALCIUM PHOSPHATE ARTICLE,” and in U.S. patent application Ser. No. 10/773,701 to Lin et al. entitled “TETRACALCIUM PHOSPHATE (TTCP) HAVING CALCIUM PHOSPHATE WHISKER ON SURFACE AND PROCESS FOR PREPARING THE SAME,” U.S. patent application Ser. No. 10/633,511 to Lin et al. entitled “METHOD OF MAKING A MOLDED CALCIUM PHOSPHATE ARTICLE,” and Int'l Patent Appl. Publ. No. WO 2005/016616 to Lin et al. entitled “METHODS FOR PREPARING MEDICAL IMPLANTS FROM CALCIUM PHOSPHATE CEMENT AND MEDICAL IMPLANTS.” The above-cited patent references are commonly assigned with the present invention and the contents thereof are hereby incorporated by reference as though fully set forth herein. Calcium phosphate cements may be formed from acidic calcium phosphates (e.g., calcium phosphates having a calcium to phosphorous ratio of less than 1.33), basic calcium phosphates (e.g., calcium phosphates having a calcium to phosphorous ratio of greater than 1.33) or combinations of acidic and basic calcium phosphates. The presently described CPCs may optionally include one or more bioactive compositions dispersed or dissolved therein, such as are described in detail below.

### Therapeutic Compositions

In some embodiments, incorporating one or more therapeutic agents into the subject compositions may enhance the biocompatibility and/or therapeutic utility of the composition.

In an embodiment, therapeutic agents may include one or more growth factors or polypeptides. The inclusion of one or more of such factors with the implant in situ may accelerate healing, vascularization, tissue and cellular infiltration of the composition. Numerous growth factors suitable for inclusion with the present embodiments are known to practitioners of ordinary skill in the art including any one of a number of polypeptide growth factors known for their ability to induce tissue or wound healing. By way of non-limiting example, growth factors or polypeptides suitable for inclusion in the presently described embodiments include, but are not limited to, osteogenin, Insulin-like Growth Factor (IGF)-1, Transforming Growth Factor (TGF)-β1, TGF-β2 TGF-β3, TGF-β4, TGF-β5, osteoinductive factor (OIF), basic Fibroblast Growth Factor (bFGF), acidic Fibroblast Growth Factor (aFGF), Platelet-Derived Growth Factor (PDGF), vascular endothelial growth factor (VEGF), Growth Hormone (GH), and osteogenic protein-1 (OP-1). In certain embodiments, growth factors belonging to the Bone Morphogenic Protein (BMP) family of growth factors, which include, but are not limited to, BMP-1, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15, bone matrix proteins (e.g., alkaline phosphatase, osteocalcin, bone sialoprotein (BSP) and osteocalcin in secreted phosphoprotein (SPP)-1, type I collagen, type IV collagen, fibronectin, osteonectin, thrombospondin, matrix-gla-protein, SPARC, alkaline phosphatase and osteopontin).

Therapeutic agents may, in some embodiments, may further include pharmacologically active compounds that do not act locally to stimulate bone growth and healing, but that may nonetheless be therapeutically advantageous in certain applications, such as, for example, antibiotic and or analgesic agents. Exemplary analgesic agents suitable for use herein include, but are not limited to, norepinephrine, bupivacaine, ropivacaine, 2-chloroprocaine, lidocaine, mepivacaine, ropivacaine, mepivacaine, benzocaine, tetracaine, dibucaine, cocaine, prilocaine, dibucaine, procaine, chloroprocaine, prilocaine, mepivacaine, etidocaine, tetracaine, xylocaine, morphine, fentanyl, alphaxalone and active analogs, 5-alpha-pregnane-3 alpha-21-diol-20-one (tetrahydro-deoxycorticosterone or THDOC), allotetrahydrocortisone, dehydroepiandrosterone, benzodiapenes, nifedipine, nitrendipine, verapamil, aminopyridine, benzamil, diazoxide, 5,5 diphenylhydantoin, minoxidil, tetrethylammonium, valproic acid, aminopyrine, phenazone, dipyrone, apazone, phenylbutazone, clonidine, taxol, colchicines, vincristine, vinblastine, levorphanol, racemorphan, levallorphan, dextromethorphan, cyclorphan, butorphanol, codeine, heterocodeine, morphinone, dihydromorphine, dihydrocodeine, dihydromorphinone, dihydrocodeinone, 6-desoxymorphine, heroin, oxymorphone, oxycodone, 6-methylenedihydromorphine, hydrocodone, hydromorphone, metopon, apomorphine, normorphine, N-(2-phenylethyl)-normorphine, etorphine, buprenorphine, phenazocine, pentazocine and cyclazocine, meperidine, diphenoxylate, ketobemidone, anileridine, piminodine, fentanil, ethoheptazine, alphaprodine, betaprodine, 1-methyl-4-phenyl-1,2,5,6-tetrahydropyridine (MPTP), loperamide, sufentanil, alfentanil, remifentanil, lofentanil, 6,7-benzomorphans, ketazocine, aryl-acetamides, U-50,488, spiradoline (U-62,066), enadoline (CI-977), asimadoline, EMD-61753, naltrexone, naltrindole.

Exemplary antibiotic agents include, but are not limited to, tylosin tartrate, tylosin, oxytetracycline, tilmicosin phosphate, ceftiofur hydrochloride, ceftiofur sodium, sulfadimethoxine cefamandole, tobramycin, penicillin, cefoxitin, oxacillin, vancomycin, cephalosporin C, cephalexin, cefaclor, cefamandole, ciprofloxacin, bisphosphonates, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericine B, ketoconazole, fluconazole, pyrimethamine, sulfadiazine, clindamycin, lincomycin, pentamidine, atovaquone, paromomycin, diclarazaril, acyclovir, trifluorouridine, foscarnet, penicillin, gentamicin, ganciclovir, iatroconazole, miconazole, Zn-pyrithione.

### EXAMPLES

The following will serve to illustrate, by way of one or more examples, systems and methods for preparing gel polymer compositions according to some embodiments, The examples below are non-limiting and are intended to be merely representative of various aspects and features of certain embodiments. Although methods and materials similar or equivalent to those described herein may be used in the application or testing of the present embodiments, suitable methods and materials are described below.

### Example 1

### Preparation of Calcium Phosphate Cement Component

### 1a) Preparation of Tetracalcium Phosphate

A Ca(PO₄)₂0 blend with CaO (TTCP) powder was prepared by mixing CaCO₃ powder with CaHPO₄ (DCPA) uniformly in ethanol followed by heating to dry, The mixing ratio of CaCO₃ powder to CaHPO₄ powder was 0.809 (weight ratio), and the powder was heated to 1400° C. to allow the two powders to react to form TTCP in combination with approximately 3% of CaO (by weight).

### 1b) Preparation of Cement Powder

The TTCP powder was ball milled and sieved then blended with ball milled DCPA powder in a ball mill. The blending ratio of TTCP powder to DCPA powder was 2.7 (weight ratio). The resultant powder mixture was added to cold water then dried. The resultant powder was mixed with approximately 0.02M phosphoric acid solution then dried. The mixing ratio of solution to powder blend is 0.32 mg/ml. The resultant powder was ball milled for approximately 0.5 hours then a portion of such powder was ball milled in ethanol for approximately 3 hours then dried and sieved.

### 1c) Preparation of Sterile Cement in Syringe

1.5 g of cement powder was placed in a syringe and sterilized with radiation.

### Example 2

### Preparation of Gel Component

### 2a) Preparation of Gel

Sodium carboxymethylcellulose (NaCMC) with high viscosity (1500-3000 cps at 1% aqueous gel) was mixed with glycerol until dispersed. The mixing ratio of NaCMC to glycerol was 0.08 (weight ratio). Water was added to the NaCMC and glycerol mixture and mixed for 10 minutes to form a gel. The mixing ratio of water to NaCMC plus glycerol was 2:1 (weight ratio), The gel was allowed to rest for more than approximately 1.5 hours. Sodium phosphate solution was prepared by mixing 7.24 g of NaH₂PO₄.H₂O and 4.69 g of Na₂HPO₄.7 H₂O with 143.27 g water until dissolved. Sodium phosphate solution was mixed with the gel for 20 minutes.

### 2b) Preparation of Sterile Gel in Syringe

3.0 g of gel with sodium phosphate was placed in a syringe and heat sterilized.

### Example 3

### Preparation of Dispersant Component (in Syringe and Sterile) 1.5 g of Glycerol was Placed in a Syringe and Heat Sterilized

### Example 4

### Preparation of Tissue Augmentation Material

The finished syringe component of example 1 was attached to the filled syringe of example 3 and mixed until visually dispersed. The resultant combination of cement and glycerol was pushed into one syringe and attached to the syringe of example 2 and mixed until visually dispersed. The resultant mixture formed a tissue augmentation device.

### Example 5

### In Vitro Performance of Tissue Augmentation Device

The tissue augmentation material of example 4 was injected through 27 gauge needle for over 30 minutes at ambient room temperature.

The tissue augmentation material of example 4 forms a cohesive bolus after curing 20 minutes in Hank's solution at 37 C.

In this patent, certain U.S. patents, U.S. patent applications, and other materials (e.g., articles) have been incorporated by reference, The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

What is claimed is:

1. A self-setting injectable composition comprising:
cement particles capable of undergoing a cementing reaction when contacted with a suitable setting liquid; and
at least one crosslinkable polymer gel, wherein said polymer gel is capable of undergoing ionic crosslinking in the presence of multivalent ions;
wherein the composition becomes substantially non-dispersive in situ.

2. The composition of claim 1, wherein the composition can be injected through a 20 to 30 gauge needle.

3. The composition of claim 1, wherein the composition remains injectable for at least 30 minutes.

4. The composition of claim 1, wherein the cement particles comprise about 0.1 to about 30 wt. % of the composition.

5. The composition of claim 1, wherein the cement particles comprise about 5 to about 25 wt. % of the composition.

6. The composition of claim 1, wherein the average diameter of the cement particles is up to about 90 μm.

7. The composition of claim 1, further comprising a buffering agent.

8. The composition of claim 7, wherein the buffering agent comprises a phosphate salt.

9. The composition of claim 7, wherein the buffering agent comprises sodium phosphate.

10. The composition of claim 9, wherein sodium phosphate comprises between about 0.1 to about 5 wt. % of the composition.

11. The composition of claim 1, wherein the cement particles are selected from the group consisting of calcium phosphate, magnesium phosphate, strontium phosphate, calcium aluminate, calcium sulfate, and calcium silicate aluminate.

12. The composition of claim 1, wherein the crosslinkable polymer gel is selected from the group consisting of cellulose, agarose, agar, agar methylcellulose, hydroxypropyl cellulose, hydroxypropyl methylcellulose, ethylcellulose, carboxyethyl cellulose, microcrystalline cellulose, oxidized cellulose, sodium carboxymethylcellulose, dextran, carboxymethyl dextran, chitosan, chitin, carboxymethyl chitin, hyaluronic acid, sodium hyaluronate, pectin, alginate, carrageenan, starch, polyuronic acids and their biocompatible salts and copolymers, polymannuronic acid, polyglucuronic acid, polyanhydroglucuronic acid, polyguluronic acid, glycosaminoglycans, heparin, heparin sulfate, and chondroiton sulfate, polyalkalene oxides and poly acids, polyethylene glycol, polyethylene oxide, polypropylene glycol, polypropylene oxide, propylene glycol alginate, polyacrylates and their acids, polylactates and their acids, polyglycolates and their acids, polymethacrylates and their acids, polymethylmethacrylates and their acids, polyterephthalic acid, polyhydroxybutyric acid, polyphosphoric acid, polystyrenesulfonic acid, polyamino acids, nonionic block copolymers, poloxamers, copolymers of acrylamide, methacrylamide, butylacrylate, maleinanhydride, and methylmetacrylate such as polyacrylamide, hydroxyethylmethacrylate, hydroxypropylmetacrylamide, peptides, gelatine, protamine, and fibrinopeptide, non-resorbable biocompatible polymers, polyethylene, polypropylene, fluoropolymers, polytetrafluoroenthylene, perfluoroalkoxy, fluorinated ethylene propylene, polymerized methyl-, ethyl-, and phenol-siloxanes, polyetheretherketone.

13. The composition of claim 1, further comprising a source of ions selected from the group consisting of biocompatible inorganic compounds of anions, phosphates, chlorides, sulfates, carbonates, ammoniums, oxides, hydroxides neutralized with suitable metal cations, sodium, potassium, calcium, magnesium, strontium, barium, lithium, beryllium, aluminum, iron, hydrogen, polycations, polyanions, mono-, di-, and tricarboxylic acids and their metal salts, and polycations and polyanions polylysine, polyarginine, and chitosan.

14. The composition of claim 1, further comprising at least one of a dispersant, a thickener, or a time-release agent.

15. The composition of claim 14, wherein said at least one dispersant, thickener, or time-release agent is selected from the group consisting of glycerol, glycol, erythritol, arabitol, xylitol, mannitol, sorbitol, isomalt, maltitol, lactitol, and polyvinyl alcohol, monosaccharides and disaccharides.

16. The composition of claim 14, wherein said dispersants, thickeners, or time-release agents comprise less than about 25 wt. % of the composition.

17. The composition of claim 1, further comprising at least one therapeutic agent.

18. The composition of claim 17, wherein said therapeutic agent is selected from the group consisting of analgesics and anesthetics. lidocaine, antiinflammatories, ibuprofen, ketoprofen, aspirin, steroids, triamcinolone, antibiotics, antihistamines, synthetic and autologous soft and hard tissue inductive growth factors, bone morphogenic proteins (BMP), chemotherapy agents, and neurotoxic analgesics.

19. A self-setting injectable composition comprising:
a poorly soluble source of inorganic ion; and
at least one crosslinkable polymer gel, wherein said polymer gel is capable of undergoing ionic crosslinking in the presence of multivalent ions;
wherein the composition becomes substantially non-dispersive in situ.

20. A self-setting injectable polymeric composition comprising:
at least one crosslinkable polymer gel;
at least one covalent or ionic crosslinking agent; and
a polyol.
